(19) United States
(12) Patent Application Publication
(10) Pub. No.: US 2024/0341339 A1
(43) Pub. Date: Oct. 17, 2024

(54) FRESH CACAO POD HUSK DERIVED PECTIN, METHOD OF ITS PREPARATION AND ITS USE IN FOOD, PHARMACEUTICAL AND COSMETIC COMPOSITIONS

(71) Applicant: CABOSSE NATURALS NV, Halle (BE)

(72) Inventor: Gabriele KOPP, Halle (BE)

(21) Appl. No.: 18/701,392

(22) PCT Filed: Oct. 17, 2022
(86) PCT No.: PCT/EP2022/078874

§ 371 Date: Apr. 15, 2024

(30) Foreign Application Priority Data
Oct. 18, 2021 (BE) 2021/5817

(51) Int. Cl. A23L 29/231; A23C 9/13; A23C 9/137; A23G 1/40; A23G 1/48; A23J 3/22; A23L 29/10; A61K 8/73; A61K 47/36

(52) U.S. Cl. CPC A23L 29/231; A23C 9/1307; A23C 9/137; A23G 1/40; A23G 1/48; A23J 3/227; A23L 29/10; A61K 8/73; A61K 47/36

(57) ABSTRACT

The invention concerns fresh cacao pod husk derived pectin, preferably characterized in a luminosity parameter L of at least 40; a water holding capacity of at least 20.0 g water/g; a molecular weight >200 kDa for at least 40 wt % of the dry extract; a storage modulus of 70 to 120 Pa measured in an emulsion at a strain amplitude of 1%; and a viscosity of 4000 to 7000 cP at 20° C. in a 5% mixture with water at a shear rate of 1/s.

The invention also concerns a method for preparing said pectin, comprising a hot water extraction from fresh cacao husks, and the use of said pectin fiber in food, cosmetics and pharmaceutical products.

### FIELD OF THE INVENTION

The present invention relates to cacao pod husk derived pectin obtainable by processing fresh cacao pod husks substantially devoid from cacao beans, wherein said pectin is characterized by a high galacturonic acid content, low degree of methyl esterification, high degree of acetylation, a high water holding capacity, a high luminosity parameter L, a high molecular weight, a high viscosity and a high storage modulus. In a second aspect, the present invention also relates to a method for extraction of said pectin. In a third aspect, the present invention relates the use of said cacao pod husk derived pectin for applications in food, cosmetic and pharmaceutical compositions.

### BACKGROUND

Theobroma cacao L., also known as a cacao tree, is a small tree in the family Malvaceae. A cacao tree produces about 20 usable pods a year. The cacao pods contain seeds, also known as cacao beans, surrounded by pod husks, placenta and pulp. The cacao seeds are used as cacao mass, powder or butter in various food applications. Given the value and great demand of the cacao seeds, the harvesting and processing of cacao is mainly focused on minimizing damage to the cacao seeds, while very little care and attention is paid to the pulp, placenta and pod husks surrounding the seeds. Traditionally, cacao pods are removed from the trees and are immediately cut in half using machetes. The seed bunch is saved, while the husk and stem are discarded.

Despite the importance of cacao as an agricultural export commodity, only approximately 10% of the gross weight of cacao pod is utilized for cacao mass, cacao powder and cacao butter production, while the majority of the total pod weight (cacao pulp, cacao placenta and cacao pod husks) is discarded as cacao waste.

Cacao pod husks represent the biggest part of cacao byproducts obtained during cacao bean production. For each ton of dried beans produced, about 16 tons (on a fresh weight basis) of cacao pod husks are left to decompose in plantations, thereby representing a serious disposal problem. The proportion of the cacao pod husk in the pod mass is in the range 68-75 wt %. Therefore, efforts have been made to convert the husk into a beneficial byproduct. The relatively high potassium content allows partial utilization of cacao pod husks as a soil fertilizer. However, rotting pods rapidly become a source of infection with microorganisms such as black pod rot. Therefore, most of the cacao pod husks are burned or buried.

Another way to utilize cacao husks is to use them as a source of insoluble and soluble fiber. Insoluble fiber such as cellulose, hemicellulose and lignin represent on average 60-70% of total fiber in cacao husks, from which 10-12% is soluble fiber.

Pectin is a water-soluble fiber and complex polysaccharide found naturally in higher plants. Pectin consists of mainly galacturonic acid units linked by α-(1,4) linkages. Pectin is widely used for its gelling properties in food, cosmetic and pharmaceutical industries. In the food industry, pectin is extracted mainly from byproducts such as citrus peel, apple pomace and sugar beet pulp.

Several studies disclosed the utilization of the cacao husks as a source of pectin.

Chan & Choo, Food Chemistry, 2013, 141, 3752-3758, investigated the effects of extraction conditions, such as temperature, extraction time and substrate on yields and properties of pectin isolated from dry cacao pod husks. Pectin was extracted using water, citric acid at pH 2.5 or 4.0, or hydrochloric acid at pH 2.5 or 4.0. It was revealed that temperature, extraction time and substrate-extractant ratio affected the yields, galacturonic acid contents, degrees of methyl esterification (DM) and degrees of acetylation (DA) of the extracted pectins. The yields and galacturonic acid contents of the extracted pectins ranged from 3.38-7.62% to 31.19-65.20%, respectively. The DM and DA of the extracted pectins ranged from 7.17-57.86% to 1.01-3.48%, respectively. The highest yield of pectin (7.62%) was obtained using citric acid at pH 2.5 [1:25 (w/v)] at 95° C. for 3.0 h. The highest galacturonic acid content (65.20%) in the pectin was obtained using water [1:25 (w/v)] at 95° C. for 3.0 h.

Vriesmann & Petkowicz, International Journal of Biological Macromolecules, 2017, 101, 146-152, disclosed dry cacao pod husks as a source of low-methoxyl, highly acetylated pectin. Said pectin is characterized by a gelling capacity in acidic media. Aqueous nitric acid was used as an extracting solvent in an optimized process of pectin extraction for a short extraction time (30 min), at pH 3.5, and temperature of 100° C. The mean experimental extraction yield was 10.7%+0.26 calculated on dry cacao pod husk weight. Galacturonic acid (59.2%) was reported as a predominant residue of reported pectin, followed by significant proportions of rhamnose (11.6%) and galactose (20.3%). The degree of methyl-esterification (DM) was 41.0% and the degree of acetylation (DA) was 17.6%.

Similarly, Vriesmann, Teófilo & Petkowicz, Food Science and Technology, 2012, 49, 108-116, describe different extraction techniques from dried cacao pod husks.

In PH12018000051 cacao pod husk is dried and milled to reduce size prior to further processing.

WO2020038905 describes a shredding of the cacao pod husk and drying at 55° C. A hammer mill further reduces the particle size.

BE1027212 describes pectins obtained from dry cacao pod husk. The cacao pod husk was dried until the moisture concentration was less than 10% by weight, preferably less than 5% by weight.

The food, cosmetic and pharmaceutical industries require new products with different characteristics. However, some above-mentioned disclosures are aiming at methods of obtaining pectin of physio-chemical and technological characteristics which limit the use of such pectin in food products. Furthermore, some of the disclosed processes are characterized by using non-green extraction solvents or/and long and severe extraction conditions, which limits their industrial applicability.

The present invention aims to resolve at least some of the problems related to processing cacao pod husks. The invention thereto aims to provide an efficient utilization of cacao pod husks for multiple purposes.

### SUMMARY OF THE INVENTION

The present invention and its embodiments serve to provide a solution to one or more of the above-mentioned problems referred to an excessive cacao pod husk waste and improved characteristics of the derived products.

Cacao pod husks are rich in dietary fiber and therefore they can be used as a cheap and abundant source for dietary fiber extraction. Pectins derived from fresh cacao pod husk obtained according to the present invention are of such physio-chemical characteristics that they are suitable for use as an ingredient in food, cosmetics and pharmaceutical products.

To this end, the present invention relates, in a first aspect, to the utilization of the discarded cacao pod husks in order to obtain cacao pod husk derived pectin according to claims 1-7. The fresh cacao pod husk derived pectin is preferably characterized:
a degree of acetylation of 30 to 50 wt % of dry extract;
a degree of methylation of 10 to 30 wt % of dry extract;
a galacturonic acid content of at least 40 wt % of ash-free dry matter;
a water holding capacity of at least 20.0 g water/g;
a luminosity parameter L of at least 40;
a molecular weight >200 kDa for at least 40 wt % of the dry extract;
a storage modulus of 70 to 120 Pa measured in an emulsion consisting of 15 g pectin, 200 g oil, 285 g water at 20° C. and at a strain amplitude of 1%; and
a viscosity of 4000 to 7000 cP at 20° C. in a 5% mixture with water at a shear rate of 1/s.

In another aspect, the present invention is directed to a method according to claim 8. More particularly, the method as described herein provides the preparation of said cacao pod husk derived pectin in a process comprising hot water extraction from cacao husks, wherein the pH upon completion of the extraction is between pH 3.0 and pH 5.0, wherein the temperature for the hot water extraction is between 60-80° C.

The methods of invention have further advantages of minimal processing times and minimal use of non-natural ingredients, leading to a clean label of the products comprising cacao pod husk derived pectin of the present invention. The said method enables obtaining said pectin in high yield, wherein said pectin is characterized in high content of galacturonic acid, low degree of methyl esterification, which is particularly important for the food industry, and a high degree of acetylation, which has an effect on the surface activity, emulsion stability, and viscosity of the product comprising said pectin.

In another aspect, the present invention relates to a use according to claim 9. Cacao pod husk derived pectin obtained according to the present invention is a suitable dispersion stabilizer in a food product. Preferably, the food product is a protein food product, starch-containing food product or a confectionery product.

In another aspect, the present invention relates to a use according to claim 11. Cacao pod husk derived pectin obtained according to the present invention is suitable to be used as an O/W or W/O emulsifier in food, cosmetic and pharmaceutical products.

Said pectin is very effective in stabilizing the oil in water (O/W) emulsions both at high and low concentrations. Said pectin showed a very good solubility in water and has shown to be a very efficient emulsifying agent which keeps emulsions stable for a couple of days at room temperature. This activity was comparable or even better than the activity of a reference emulsifier, such as lecithin.

Moreover, said pectin efficiently enhanced viscosity of different solutions even when applied in low concentrations. Applications of said pectin in food products are numerous, due to its good water solubility and easy application. Cacao pod husk derived pectin can be also used in cosmetics products, such as, but not limited to lotions, creams, shower gels, balms, after shave creams and gels, and the like. When used in said products, said cacao pod husk derived pectin is used in a concentration 0.05 to 50.00 wt %.

In another aspect the present invention relates to a use according to claim 14. Cacao pod husk derived pectin is used as an elastic agent in food, cosmetic and pharmaceutical products.

In a final aspect the present invention relates to a use according to claim 15. Cacao pod husk derived pectin is used in plant-based meat or in regular meat.

### BRIEF DESCRIPTION OF THE FIGURES

FIG. 1 shows a standard vegan burger.

FIG. 2 shows a standard burger with 2% insoluble fiber from dried husk.

FIG. 3 shows a standard burger with 2% pectin from fresh husk.

### DETAILED DESCRIPTION OF THE INVENTION

The present invention concerns solving some of the problems related to cacao pod husk utilizations. The present invention discloses cacao pod husk derived pectin obtainable from cacao pod husks which are substantially devoid from cacao beans. The present invention also discloses methods of extraction of said pectin and its further use for various purposes in food, pharmaceutical and cosmetic products.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise.

“Comprise”, “comprising” and “comprises” and comprised of” as used herein are synonymous with “include”, “including”, “includes” or “contain”, “containing”, “contains” and are inclusive or open-ended terms that specify the presence of what follows e.g. component and do not exclude or preclude the presence of additional, non-recited components, features, elements, members, steps known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in sequences other than those described or illustrated herein;

The expression “% by weight”, “weight percent”, “% wt” or “wt %”, here and throughout the description unless otherwise defined, refers to the relative weight of the respective component based on the overall weight of the formulation. The galacturonic acid content is expressed throughout the description in wt % of ash-free dry matter, i.e., on an ash-free dry matter content.

Reference throughout this specification to “one embodiment” or “an embodiment” means that a particular feature, structure or characteristic described in connection with the embodiment is included in at least one embodiment of the present invention. Thus, the appearance of phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

It should be noted that the terms “cacao” and “cocoa” are both often used to refer to the same items. For example, cacao beans are often referred to as “cocoa” beans. In the present application, the term “cacao” is generally used to refer to the used materials originating from Theobroma cacao.

The term “cacao pod” refers to a cacao fruit, which comprises cacao pod husks, cacao pulp, cacao placenta and cacao beans.

The term “fresh cacao pod husk” or “fresh cacao husk” or “fresh husk” refers to a cacao fruit husk, with a moisture content above 50 wt % and preferably above 80 wt %.

The term “dry cacao pod husk” or “dry cacao husk” or “dry husk” refers to a cacao fruit husk, with a moisture content below 20 wt %, preferably below 5 wt. % and more preferably below 3 wt. %.

The term “dietary fiber”, as used herein, means the sum of the indigestible carbohydrate and carbohydrate components of food, including cellulose, lignin, hemicelluloses, pentosans, gums and pectins.

The term “protein food products”, as used herein, means the food products containing animal or vegetable proteins. Said products include a variety of food products, for example, beverages containing animal or vegetable proteins such as milk, soybean milk or the like, acidic dairy beverages obtained from dairy products, frozen desserts such as acidic ice cream, frozen yogurt and the like or milk containing frozen desserts such as ice cream, chocolate, acidic desserts such as puddings or bavarois, as well as coffee beverages, cacao beverages, lactic acid bacteria beverages (containing live bacteria, or sterilized types), fermented milk (solid or liquid), and the like. Animal or vegetable proteins including cow milk, sheep milk, skimmed milk, and soybean milk, whole milk powder forms of such milk, skim milk powder, soybean milk powder, sugar-added milk, milk concentrates, processed milk fortified with minerals such as calcium or vitamins, fermented milk, and proteins derived therefrom. Fermented milk is milk obtained by sterilizing the aforementioned animal or vegetable proteins and then adding a lactic acid bacteria starter for fermentation, and if desired, it may be powdered or the like may be added thereto, or it may be heat sterilized. Said protein food products can be acidified using citrus fruit juices or other fruit juices, or organic acids such as citric acid or lactic acid or inorganic acids such as phosphoric acid.

The term “starch-containing food products”, as used herein, means the food products prepared by cooking, steaming or boiling dough prepared using wheat flour as the raw material. Said starch containing food products comprise, but are not limited to cookies, biscuits, crackers, sponge cakes, various types of bread, or dough composed mainly of starch, such as rice cakes.

“About” as used herein referring to a measurable value such as a parameter, an amount, a temporal duration, and the like, is meant to encompass variations of +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

The invention relates to fresh cacao pod husk derived pectin with a water holding capacity (WHC) of at least 5.0 g water/g, preferably 10 g water/g.

In a first aspect, the invention relates to fresh cacao pod husk derived pectin with a water holding capacity (WHC) of at least 20.0 g water/g, preferably 23 g water/g. The water holding capacity of a material is its capacity to trap and hold water in and on the surfaces and the pores of the particles. To assess the water holding capacity of a sample, 0.5-1.0 g is mixed in water (50 g) at 20° C. The suspension is then centrifuged at 3000 g for 30 minutes and the supernatant decanted into a pre-weighed aluminum moisture tin. The weight of sediment is measured to assess the water holding capacity. The water holding capacity equals the weight of the pellet minus the sample weight prior to mixing with water followed by a division of this difference by said sample weight.

In a further embodiment, the assessment of the WHC can also include a step to check if some of the pectins dissolved in the water. In an embodiment, the pellet is dried again after weighing and the difference with the initial sample weight is corrected for. In another embodiment, the decanted water is evaporated, the dissolved products are dried at 105° C. and weighted. The WHC is corrected based on the weight of the dissolved products.

The WHC of fresh cacao pod husk derived pectin is at least two times higher compared to dry cacao pod husk derived pectin, preferably at least 3 times, more preferably at least 4 times, more preferably at least 5 times, more preferably at least 7 times, more preferably at least 10 times. The WHC of fresh cacao pod husk derived pectin is two to twenty times higher compared to dry cacao pod husk derived pectin, preferably at least 3 times, more preferably at least 4 times, more preferably at least 5 times, more preferably at least 7 times, more preferably at least 10 times.

Fresh cacao pod husk derived pectin a pectin with a WHC at least two times higher compared to the WHC of pectin derived from dry cacao pod husk, preferably at least 3 times, more preferably at least 4 times, more preferably at least 5 times, more preferably at least 7 times, more preferably at least 10 times.

In a second aspect, the invention relates to fresh cacao pod husk derived pectin with a molecular weight >200 kDa for at least 40 wt % of the dry extract, preferably 50 wt % of the dry extract has a molecular weight >200 kDa. The molecular weight referred to throughout the present specification is the value measured by gel filtration HPLC, with standard pullulan as the reference substance.

The majority, i.e. more than 50 wt %, of pectins derived from dried cacao pod husk have a molecular weight of less than 10 kDa. The drying step unexpectedly seems to reduce the stability of the pectin fibers. The water holding capacity is linked to molecular size which is dependent on the process conditions to get the pectin from the husk.

In a third aspect, the invention relates to fresh cacao pod husk derived pectin with a storage modulus of 70 to 120 Pa measured in an emulsion consisting of 15 g pectin, 200 g oil, 285 g water at 20° C. and at a strain amplitude of 1%. Preferably the storage modulus is between 80 and 115 Pa. The viscoelastic properties of this oil-in-water emulsions sample, was measured by performing an amplitude strain sweep test. This test is conducted using a rheometer, fitted with a cone and plate geometry (diameter 50 mm and gap of 0.1 mm). The applied strain used was 0.01 to 100%, which was ramped from low to high, logarithmically increasing 5 points per decade (21 points in total). A constant frequency of 1 Hz and a measurement temperature of 20° C. were applied. The measurements were performed in duplicate on each emulsion sample after one day post-production.

In a fourth aspect, the invention relates to fresh cacao pod husk derived pectin with a luminosity parameter L of at least 40, preferably at least 45. Color was measured in CIELab space coordinates, L (lightness: 0=black, 100=white), a (−a=greenness, +a=redness) and b (−b=blueness, +b=yellowness) in a Cr- 5 colorimeter.

In a fifth aspect, the invention relates to fresh cacao pod husk derived pectin characterized in a viscosity of 4000 to 7000 cP at 20° C. in a 5% mixture with water at a shear rate of 1/s, preferably between 5000 and 6000 cP. To measure the viscosity, the pectins were mixed with distilled water using a magnetic stirrer to obtain a 5% mixture. Rheology measurements were performed using Rheometer. Parallel plates (PP50) were used at a gap of 0.5 mm. The viscosity was measured using rotational rheometry over the shear rate range 0.1-100/s at 20° C.

The water holding capacity of at least 20.0 g water/g, molecular weight >200 kDa for at least 40 wt % of the dry extract, storage modulus of 70 to 120 Pa, luminosity parameter L of at least 40 and viscosity of 4000 to 7000 cP, indicate that the fresh husk derived pectins are significantly different dry husk pectins. These characteristics indicate an improved applicability and value of the product.

In an embodiment, the invention relates to fresh cacao pod husk derived pectin according to the first aspect and one or more of the second, third, fourth and fifth aspect.

In an embodiment, the invention relates to fresh cacao pod husk derived pectin according to the first aspect and second aspect. In an embodiment, the invention relates to fresh cacao pod husk derived pectin according to the first aspect and third aspect. In an embodiment, the invention relates to fresh cacao pod husk derived pectin according to the first aspect and fourth aspect. In an embodiment, the invention relates to fresh cacao pod husk derived pectin according to the first aspect and fifth aspect. In an embodiment, the invention relates to fresh cacao pod husk derived pectin according to the first aspect, second aspect and third aspect. In an embodiment, the invention relates to fresh cacao pod husk derived pectin according to the first aspect, second aspect and fourth aspect. In an embodiment, the invention relates to fresh cacao pod husk derived pectin according to the first aspect, second aspect and fifth aspect. In an embodiment, the invention relates to fresh cacao pod husk derived pectin according to the first aspect, second aspect, third aspect and fourth aspect. In an embodiment, the invention relates to fresh cacao pod husk derived pectin according to the first aspect, second aspect, third aspect and fifth aspect. In an embodiment, the invention relates to fresh cacao pod husk derived pectin according to the first aspect, second aspect, third aspect, fourth aspect and fifth aspect. In an embodiment, the invention relates to fresh cacao pod husk derived pectin according to the first aspect, third aspect, fourth aspect and fifth aspect. In an embodiment, the invention relates to fresh cacao pod husk derived pectin according to the first aspect, third aspect and fifth aspect. In an embodiment, the invention relates to fresh cacao pod husk derived pectin according to the first aspect, third aspect and fourth aspect. In an embodiment, the invention relates to fresh cacao pod husk derived pectin according to the first aspect, fourth aspect and fifth aspect.

In an embodiment, fresh cacao pod husk derived pectin, according to one or more of the previous aspects, has a degree of acetylation of 30 to 50 wt % of dry extract; a degree of methylation of 10 to 30 wt % of dry extract; and a galacturonic acid content of at least 40 wt % of ash-free dry matter.

In another aspect, the invention also relates to a method for the preparation of fresh cacao pod husk derived pectin according to any of the previous aspects comprising a hot water extraction, wherein the pH upon completion of the extraction is between pH 3.0 and pH 5.0, and wherein the temperature for the hot water extraction is between 6° and 80° C., preferably 70° C. In an embodiment, the pH value is adjusted by using acetic, citric, lactic, malic, tartaric, hydrochloric, nitric, oxalic, phosphoric and sulfuric acid.

The present invention discloses methods of producing cacao pod husk derived pectin with several benefits for further use in food and related products. The pectin obtained by the present invention is characterized by the high level of galacturonic acid, and a particularly low methyl esterification degree and high acetylation degree. The methods disclosed in the present invention are optimized to maximize the extraction yield, minimize processing time, number of processing steps and use of non-green solvents and reactants. Thus, cacao pod husk derived pectin obtained according to the present invention is a green product with a clean label. Furthermore, cacao pod husk derived pectin can substitute genetically modified soy pectin in many products, preferably food products. Sunflower derived pectin, lecithin or artificial pectin substitutes can also be replaced with the pectins in the present invention.

Cacao pods are first inspected and brushed. Only the healthy pods will be subjected to a pre-treatment and processed further, i.e., being disinfected and washed.

The first step of the process for complete utilization of cacao byproducts according to the present invention is pre-treatment of cacao pod husks in order to debacterize the pod surface. This step is required if the husks are not further processed within 10 days, preferably 5 days and more preferably 3 days. Because cacao pods are perishable products, thus rotting pods rapidly become a source of infection with microorganisms. Therefore, it is necessary to ensure their microbiological quality prior to production process. For such pre-treatment, according to the present invention, unopened cacao pods are treated with a preserving agent to avoid enzymatic deterioration, for a period of time sufficient to neutralize any harmful substances on the surface of the cacao fruit and stabilize lignification of the outer layer (peeling) of the cacao fruit. Almost any known preserving agent effective in a low concentration can be employed in this process, such as sorbic acid and salts thereof, sulfur dioxide, chlorine, chlorides, nitrates, hypochlorites, preferably those of the alkali metals, disulfites, ozone, methylformate, diphenyl, sodium ortho-phenyl phenolate, nitrogen trichloride and others. Typically, the preserving agent is employed in the form of an aqueous solution of a low but effective concentration. The concentration of the preserving agent depends, of course, upon its effectiveness and varies considerably. Optimum concentrations which do not detrimentally affect the quality of cacao fruit, can readily be determined by a person skilled in the art by routine experimentation. It is also possible to apply the preserving agent by exposing the unopened cacao pod husks to the vapors of vaporizable preserving agents, preferably diluted with air or inert gases. The treatment with the preserving agent varies depending upon the concentration and effectiveness of the agent and may last for a period of time between 1 hour and up to sixty days, preferably between 8 hours and 8 days although it is not limited to such a period of time. The “whole”, harvested cacao fruit is placed directly into a bath or atmosphere containing a preservative, typically at a low but effective concentration of preservative, for instance, between 0.2 and 10%, and for a period of time sufficient to neutralize any toxic substances on the surface of the fruit and to stabilize lignification of the outer layer (peeling) of the fruit. To accomplish this, it generally requires between 10 min and 3 or more months, preferably 30 min to 24 h, most preferably 30 min to 3 h of exposure to the bath or atmosphere, depending upon the effectiveness and type of preservative used. Any known preservative can be employed in this step of the process, for example, sorbic acid, sorbic acid salts, sulfur dioxide, chlorides, nitrates, chlorine, hypochlorite, formaldehyde, disulfides, ozone, methylformate, diphenyl, ammonia, sodium o-phenyl phenolate, nitrogen trichloride, and others. The preferred preserving agents are sorbic acid, sulfur dioxide, chlorine, sodium chloride and hypochlorite, as many other known agents, although useful, tend to affect the taste and nutritive value of the treated cacao pods.

According to the present invention, cacao pod husk derived pectin can be extracted from cacao pod husks with outer peel, or cacao pod husks obtained after the removal of the outer peel. If the extraction is conducted with the outer peel, a washing is required. If the outer peel is removed, no such washing is required.

In accordance with the method for processing the cacao pods disclosed in the present invention, the outer skin or peeling of the harvested cacao fruit can be optionally removed before the fruit is opened to remove the cacao beans together with mucilage pulp and placenta. If the outer peel has not been removed, the remaining fruit is neutralized and the action of enzymes on the fruit surface is reduced or completely eliminated by treating the fruit with a suitable acid. Said acid is preferably an edible acid, such as, but not limited to citric acid, malic acid, tartaric acid, ascorbic acid, and the like. Phosphoric acid and hydrochloric acid may also be used. Treatment with sulfur dioxide is also possible and frequently of considerable advantage. The amount of acid added is such that the pH-value of the treated cacao fruit is below a pH of 7.0, preferably at a pH between 5.0 and 6.0.

The following step disclosed in the present invention comprises opening of the cacao fruit and removal of cacao beans together with pulp and placenta. The opening of the cacao pods is done by a cacao pod splitting machine, or any other suitable instrument. The pod husk substantially devoid from cacao beans is shredded in any cutting machine or disintegrator to obtain shredded cacao pod husks or a pod husk paste. In an embodiment, a about 1 mm cutting head was used to conduct the shredding. Cacao pod husk flakes are then preserved using a preservation agent or at low temperatures until further processing. Preferred preservation agents are acids such as citric acid, malic acid, tartaric acid, ascorbic acid, and the like.

Recently, considerable attention has been directed towards the importance of dietary fiber. Dietary fiber is generally defined as the sum of the indigestible carbohydrate and carbohydrate components of food, including cellulose, lignin, hemicelluloses, pentosans, gums and pectins. The market for dietary fiber is highly competitive. The production of dietary fiber rich products from fruit byproducts and the potential preparation of those fibers with other associated bioactive compounds is gaining more attention. While milling and enzymatic digesting have been the main steps in obtaining high dietary fiber powders from cereals; wet milling, washing, drying and dry milling are very important in producing fibers from fruit. Milling is not needed when processing the fresh husk.

In an embodiment, the cacao pod husk paste was subjected to a prewashing in absolute or diluted ethanol, preferably ethanol 85% v/v. Optionally, this step was repeated. The excessive liquid was decanted, filtered, evaporated or decreased by any suitable method.

Alternatively, cacao pod husk paste can be treated with suitable protease, such as, but not limited to Bacillus licheniformis protease, to enhance the peptide extraction process. Said bacterial protease can be used in a concentration range determined by a skilled person, and generally is 0.1 wt % dry solids, though higher or lower concentrations are equally possible given the multiple factors available.

The solid part (cacao pod husk paste after washing and alternatively after treatment with suitable protease) was subjected to an extraction process, using hot water in acidic environment (pH<7.0), preferably at pH 2.0 to 6.5, most preferably pH 3.0 to 5.0, and even more preferably at pH 4.0. Hot water extracts obtained outside of this pH range were shown not to adequately exhibit the function important for future use according to the present invention.

Since dietary fiber extracted in the alkaline range of pH 7.0 and above has a high hemicellulose content and a low content of pectinic polysaccharides containing galacturonic acid, it cannot provide adequate dispersion stability of proteins in the weak acidic pH range above the isoelectric point. In addition, galacturonic acid methyl ester is partially decomposed while the polysaccharides themselves are decomposed by elimination. The flavor is also impaired due to reaction of sugars with the protein.

The ratio solid mater/extractant was not higher than 1:1 w/v, preferably not higher than 1:10 w/v, most preferably not higher than 1:20 w/v. The acids used for the extraction procedure can be any acid suitable for use in the food industry, such as, but not limited to acetic, citric, lactic, malic, tartaric, hydrochloric, nitric, oxalic, phosphoric and sulfuric acid. In a preferred embodiment, this acid is an organic acid such as acetic, citric, lactic, malic and tartaric, most preferably citric acid.

The extraction temperature to obtain the cacao pod husk derived pectin in the aforementioned pH range is preferably between 60-80° C. under atmospheric pressure for two hours. When the extraction is performed at a temperature of below 60° C., time is required for elution of the pectin, thus creating an economic disadvantage. On the other hand, while the extraction is completed in a shorter time with a higher temperature, an excessively high temperature will adversely affect the flavor and color while also resulting in reduced function due to the lower molecular weight of cacao pod husk derived pectin; the temperature is therefore preferably no higher than 100° C., most preferably no higher than 80° C. The lighter color obtained via the method as described in this invention results in a product with a broader application as dark colors are often not desired in products.

The extraction process is performed during the period up to 5 h, preferably 1-4 h, more preferably 1.2-3 h and most preferably 2 h. Optionally, high pressure, or any other suitable industrial method can be used to facilitate the extraction process.

In some embodiments, the extraction can, alternatively, be facilitated by the treatment with enzymes such as proteases, cellulases, hemicellulases, pectinases, amylases and the like. Acids can also be used such as citric acid, acetic acid, etc. Also, while the obtained cacao pod husk derived pectin can be dried for direct use after the extraction, in a preferred embodiment the mineral components and the residual citric acid are at least partially removed (desalted) by electrodialysis, ion-exchange resin treatment, nanofiltration, ultrafiltration, or the like. Optionally, cacao pod husk derived pectin of a more satisfactory quality can be obtained by carrying out an active carbon treatment, resin treatment, precipitation treatment with a solvent such as ethanol or isopropanol and the like. Cacao pod husk derived pectin of a more satisfactory quality can also be obtained by removal of the low molecular weight color components or foul-tasting components (purification) by UF membrane or ceramic filter separation.

After the acid extraction of the water-soluble material, the pH is not lower than 3.0, more preferably around 4.0. After cooling, residues are separated from the supernatants using centrifugation. This was done using a standard centrifuge or any other suitable system, for not shorter than 30 min. In an embodiment the residue was combined with an equivalent weight of water, or any other suitable solvent (ethanol, isopropanol, etc.) or solvent mixture, and the mixture was again centrifuged. In an embodiment, the resulting supernatant was mixed with the previous water-soluble fraction and the extract was directly lyophilized to obtain cacao pod husk derived pectin. In an embodiment, cacao pod husk derived pectin can be purified by an active carbon column or any other suitable purification process.

The following step is precipitation of cacao pod husk derived pectin using alcohol, preferably ethanol. Cacao pod husk derived pectin was mixed with said alcohol and precipitate is collected using simple decantation process or filtering system. Precipitate was collected and optionally re-washed with ethanol in concentrations 80%, 90% and 99% v/v. In a preferred embodiment after adding 85% ethanol to cacao pod husk derived pectin extract a concentration of 50% of ethanol is obtained in supernatant and pectin was precipitated. The precipitate was successively re-washed with ethanol in the same manner and air dried to obtain said pectin. The precipitation process and washing with ethanol can be repeated until a precipitate of suitable characteristics is obtained. Precipitation temperature was not higher than 85° C., preferably not higher than 70° C., most preferably the precipitation temperature is 60° C.

Alternatively, ultrafiltration can be used to further purify and up-concentrate the cacao pod husk derived pectin. In some embodiments, prior to the precipitation or ultrafiltration an additional concentration step can be conducted, to increase efficiency of the ethanol or the ultrafiltration.

Additionally, cacao pod husk derived pectin can be recovered by filtration on a suitable strainer. If necessary, said recovered cacao pod husk derived pectin can be washed and precipitated by using alcohol in the above-mentioned step.

After the drying process, the weight of obtained cacao pod husk derived pectin is measured and said cacao pod husk derived pectin is further used for preparing disclosed compositions. The total yield of obtained cacao pod husk derived pectin is not less than 3.0 wt %, preferably not less than 6.0 wt %, most preferably not less than 10.0 wt %, calculated on a dry cacao pod husk powder.

Cacao pod husk derived pectin according to the present invention comprises pectin, characterized by a low methyl esterification degree. The said degree of methyl esterification is not higher than 40%, preferably 10-30%, most preferably 10-15% of dry extract. The degree of esterification has an impact on the physical properties of pectin: emulsion formation, surface tension, stabilization of tissue, and gel characteristics.

Depending on their degree of methyl esterification (DE), pectins are referred to as high methyl esterified pectins or high metoxy pectins (DM ≥50%) or low methyl esterified (methoxy) pectins (DE<50%). High methyl esterified pectins form gels in an acidic medium (pH 2.0-3.5) if sucrose is present at a concentration higher than 55 wt %. Low methyl esterified (methoxy) pectins can gel over a larger pH range (2.0-6.0) in the presence of a divalent ion, such as calcium. In this case, less sucrose is necessary for forming the gel.

Pectins with a low degree of methyl esterification are particularly suitable for making low sugar confectionery products, such as, but not limited to, jams and different fruit preparations, but also in protein food products such as yoghurts, chocolate milk and milk beverages for use in the food industry. Thus, cacao pod husk derived pectin is characterized by a low methyl esterification degree is suitable for the low sugar confectionary products as well as protein beverages.

Cacao pod husk derived pectin according to the present invention is characterized by a high acetyl esterification degree. The said degree of acetylation is 20-60%, preferably 30-50%, most preferably 40-50% of dry extract. Acetylation, like methylation, decreases the affinity of pectin for cations, which has an impact on the gelling ability of pectin. Furthermore, it has an effect on surface activity, emulsion stability and viscosity of compositions comprising said pectin.

The content of galacturonic acid in said pectin is not less than 40 wt %, preferably not less than 50 wt %, and most preferably not less than 60 wt % of ash-free dry material.

An increase in acid strength (that is, decreasing pH) of the water extraction can affect the galacturonic content at the end of this process. Moreover, acid type and concentration affect the yield, physiochemical and functional properties of pectin. Galacturonic acid content was measured according to the Blumenkrantz method, and neutral sugars were measured by GLC as alditol acetates.

However, said cacao pod husk derived pectin is comprising galactose, rhamnose, glucose, mannose, ribose, inositol, myo-inositol, moisture (up to 20 wt %), (glucurono) arabinoxylans (GlCA, Ara and Xyl), galactan (Gal), mannan (Man). Most remarkable were the high amount of arabinoxylans (present in concentration of at least 5 wt % of disclosed cacao pod husk derived pectin) that can be used as prebiotics. The cacao pod husk derived pectin obtained by the present invention is characterized by xylooligosaccharides content of at least 1.5 wt %, preferably at least 3.0 wt % of the dry weight of cacao pod husk derived pectin. The cacao pod husk derived pectin obtained by the present invention is characterized by galactomannan content of at least 1.5 wt %, preferably at least 3.0 wt % of the dry weight of cacao pod husk derived pectin. The cacao pod husk derived pectin obtained by the present invention is characterized by glucomannan content of at least 1.5 wt %, preferably at least 3.0 wt % of the dry weight of cacao pod husk derived pectin.

Xylooligosaccharides selectively feed beneficial bacteria such as bifidobacteria and lactobacilli within the digestive tract. A large number of clinical trials have been conducted with xylooligosaccharides, demonstrating a variety of health benefits, including improvements in blood sugars and lipids, digestive health benefits, laxation, and beneficial changes to immune markers. These health benefits have typically been observed at 1-4 g/d, which is a lower dose than required for prebiotics such as fructooligosaccharides and inulin.

Glucomannan is a dietary fiber. Orally, glucomannan is used for constipation, weight loss, diabetes, high cholesterol, overactive thyroid (hyperthyroidism), high blood pressure, and stomach conditions called dumping syndrome and functional gastrointestinal disorders. In food products, glucomannan is used as a thickener or gelling agent. Glucommanan flour and powder are used in food. Similarly, galactomannans, as dietary fibers are often used in food products to increase the viscosity of the water phase.

Fresh cacao pod husk derived pectin extracted from cacao pod husks according to the invention can unexpectedly be distinguished from dry cacao pod husk derived pectin, based on several properties. The pectin extract from fresh husks has a lighter color and significantly higher texturizing performance. A similar emulsifying performance, but stronger emulsion stiffness was observed for the fresh cacao pod husk derived pectin compared to pectins derived from the dried product. The water holding capacity was several times higher for the pectins derived from fresh compared to dried husks. The water holding capacity of pectins derived according to this invention was least 20.0 g water/g pectin. Pectins from both fresh and dried cacao pod husks showed good and fasts dispersibility compared to benchmarks, a good solubility and a good oil holding capability. It was concluded that extracts from dried and fresh husks show potential to stabilize water-in-oil emulsions with both different characteristics and applications.

The present invention aims to produce a low-cost cacao pod husk derived pectin characterized by the additional health benefits (prebiotic), higher market price setting and ingredient functionality. The above-mentioned soluble fibers are particularly suitable for use in fat-based dispersions and have a low impact on adherence and graininess.

The production method as described herein, is environmentally friendlier compared to the conventional production method for pectin from dried cacao pod husk. No drying step is used to prevent spoiling nor is a steam or alkaline solution treatment required to ease the peeling. During extraction, more gentle conditions are applied such as a more neutral pH, less heating, etc. These changes further reduce the environmental impact and energy requirements.

The cacao pod husk derived pectin used according to the invention may have any molecular weight value, but it preferably has an average molecular weight of from a few tens of thousands to a few million Da, more preferably from a few tens of thousands to a few hundred thousand Da, and specifically from 20,000 to 500,000 Da. The average molecular weight referred to throughout the present specification is the value measured by gel filtration HPLC using a TSK-GEL G-5000PWXL, with standard pullulan as the reference substance. In an embodiment, to fresh cacao pod husk derived pectin with a molecular weight >200 kDa for at least 40 wt % of the dry extract, preferably >200 kDa for 50 wt % of the dry extract.

It is an object of one aspect of the present invention to provide cacao pod husk derived pectin suitable for use in protein foods and preparation methods for said foods. The present invention is aimed at providing heat-sterilized milk beverages whose milk components are stable for prolonged periods and which can be transported at ordinary temperature.

It is an object of another aspect of the invention to provide chocolate beverages with low precipitation of solids and a satisfactory state of dispersion of the fat and milk components. Preparation of chocolate beverages according the invention may be accomplished by any ordinary preparation method, using chocolate components, sweeteners and milk components as raw materials, in addition to using cacao pod husk derived pectin as a dispersion stabilizer. The chocolate components used may be any one or more selected from among cocoa powder, cacao mass, cocoa butter and cocoa butter substitute.

Any publicly known sweetener may be used, for example, one or more selected from sugars such as sucrose, glucose, fructose, isomerized sugar, rice jelly, trehalose, maltitol or sorbitol, or other sweeteners such as aspartame, stevia, glycyrrhizin, thaumatin or the like.

The cacao pod husk derived pectin may be used in an amount of about 0.05-10.00 wt %, preferably 0.10-2.00 wt % and more preferably 0.10-1.00 wt % with respect to the final food product, but these ranges are not limitative on the scope of the invention as they will vary depending on differences in composition. The protein food products may also be prepared together with conventional stabilizers, for example, polysaccharides such as pectins, water-soluble soybean polysaccharides, carboxymethylcellulose sodium, alginic acid propylene glycol ester, carrageenan, furcellan, tamarind seed polysaccharides, tara gum, karaya gum, guar gum, locust bean gum, tragacanth gum, pullulan, gelan gum, native gelan gum, gum Arabic, dextrin, cyclodextrin, agar, microcrystalline cellulose, xanthan, processed starch and the like, or hydrolysates thereof, gelatin, organic acid salts, polymerization phosphoric acid salts, emulsifiers, heat-denatured proteins and the like.

The dairy products used may be any ordinary products, and as specific examples there may be mentioned milk, whole powdered milk, powdered skimmed milk, cream, butter, whole condensed milk, condensed skimmed milk, processed milk powder and the like.

The amount of the cacao pod husk derived pectin added to a chocolate beverage is preferably 0.05-20.0 wt %, more preferably 0.1-10.0 wt % and most preferably 0.2-3.0 wt % with respect to the final beverage. The stabilizing effect may be inadequate if the amount is too small, while the influence on the viscosity of the beverage increases if the amount is too large. The pH of the chocolate beverage is preferably from pH 5.0-9.0, more preferably pH 5.5-8.0 and most preferably pH 6.0-7.5.

According to the invention, other emulsifiers and dispersion stabilizers may be used in the aforementioned chocolate beverages, regardless of whether they are in liquid form or in a powdered or paste form. Any publicly known emulsifiers or dispersion stabilizers may be used, and specifically there may be mentioned sucrose fatty acid esters, glycerin fatty acid esters, sorbitan fatty acid esters, propylene glycol fatty acid esters, polyglycerin fatty acid esters, lecithin, agar, carrageenan, furcellan, tamarind seed polysaccharides, tara gum, karaya gum, soybean hemicellulose, pectin, xanthan gum, sodium alginate, tragacanth gum, guar gum, locust bean gum, pullulan, gelan gum, gum Arabic, gelatin, casein sodium, various starches, various celluloses.

Soy lecithin has been the frontrunner for many years as a common emulsifier in many common consumer goods including chocolate, bakery and confectionery products, and even nutritional supplements. But with the growing concerns around GMO crops and allergic/intolerance reactions that surround soy in general, it urges to consider replacing this ingredient with a suitable stabilizer such as cacao pod husk derived pectin disclosed in the present invention. Fresh cacao pod husk derived pectins are suitable to replace soy lecithin in food, cosmetic and pharmaceutical products. Said food products are, but not limited to chocolate, pastry, beverages, milk beverages, yoghurt, confectionery products, and the like. The cacao pod husk derived pectin may be used in an amount of about 0.05-10.00 wt % and preferably 0.10-5.00 wt % with respect to the final food product, but these ranges are not limitative on the scope of the invention as they will vary depending on the differences of the products. Said food products may contain also other conventional stabilizers, for example, polysaccharides such as pectins, water-soluble soybean polysaccharides, carboxymethylcellulose sodium, alginic acid propylene glycol ester, carrageenan, furcellan, tamarind seed polysaccharides, tara gum, karaya gum, guar gum, locust bean gum, tragacanth gum, pullulan, gelan gum, native gelan gum, gum Arabic, dextrin, cyclodextrin, agar, microcrystalline cellulose, xanthan, processed starch and the like, or hydrolysates thereof, gelatin, organic acid salts, polymerization phosphoric acid salts, emulsifiers, heat-denatured proteins and the like.

In another aspect of the present invention, the cacao pod husk derived pectin can be used as an efficient O/W or W/O emulsifier in food, cosmetic and pharmaceutical products. The cacao pod husk derived pectin may be used in an amount of 0.05-50.00 wt %, preferably in amount 0.05-20.00 wt %, most preferably in amount of 0.10-8.00 wt % with respect to the final product. Said cacao pod husk derived pectin is effective in a relatively small amount, it is compatible with additives commonly used in O/W emulsions and it is an efficient emulsifier oven a wide pH range of the finished product. The said products are characterized by good tolerability and hypo-allergenic properties. Said cacao pod husk derived pectin is particularly suitable for cosmetic products such as, but not limited to body lotions, body and face creams, cleansing emulsions, sun block creams, body milk and the like. Cacao pod husk derived pectin according to the invention is characterized by a good skin tolerability and a pleasant feeling when applied topically. The body care products comprising said pectin are characterized by the good stability and no separation of the phases.

It is an object of another aspect of the invention to provide a coating agent which exhibits effects of glazing, protection from oxidation, shelf-life extension, increased bake stability, better coating properties and stronger sugar coating for food products, and which can be produced easily and inexpensively.

When cacao pod husk derived pectin is used as a coating agent, it is preferably added at 0.1-50 wt % and most preferably 0.5-30 wt % with respect to the total coating agent. It may also be used outside of these ranges when used in a sugar coating. Depending on the amount of sugar, a small amount of fiber may result in low coating strength or cracking and flaking of the sugar coating, while an excessive amount of fiber will tend to lengthen the drying time, and therefore the fiber is preferably used within the ranges specified above.

The cacao pod husk derived pectin may be used alone as a coating agent but, if necessary, there may also be added plasticizers, pigments, dispersing agents, solvents, taste substances, coloring agents, preservatives, de-foaming agents and the like. It may be used together with other coating agents, for example, polysaccharides such as guar gum, tragacanth gum, xanthan gum, carrageenan, tamarind gum, locust bean gum, agar, gum Arabic, processed starch, hydroxypropylmethyl cellulose and pullulan or proteins such as gelatin.

The method of using the said pectin as a coating agent may involve spraying or immersion after preparing a solution of the pectin. As apparatuses for spray coating any suitable apparatus including hi-coaters, aqua coaters, flow coaters, Spira-flows, rotor container-equipped fluidized bed apparatuses and the like can be used. For immersion, any apparatus having an immersion layer and a drier may be used.

The invention further discloses a food product comprising the cacao pod husk derived pectin according to the invention. Said food product comprises, but is not limited to a frozen confection, yoghurt, dessert, a bakery product, or a sauce.

According to the present invention said pectin is added to an ice cream to get a product which is spooned easily by a spoon, even relatively rapidly after being taken out of a freezer, or the like. When the cacao pod husk derived pectin of the invention is used as an ice-cream texturizer, said pectin is added at 0.1-25.0 wt % 0.2-10.0 wt % and most preferably 0.5-5.0 wt % with respect to total weight of the ice cream, or finished product. Cacao pod husk derived pectin may be pre-added, or it may be added together with other materials such as water and mixed therewith, and then cooked, steamed or boiled, according to the ordinary methods for various food products, to obtain different food products. In addition, the ice cream comprising the pectin of the present invention is characterized by an excellent softness in the mouth, even at a freezing temperature and excellent flavor, without the excessive addition of components having an anti-freezing effect such as salt, alcohol and the like to an ice cream mixture.

Baking stable water-based preparations are used for bakery products with the aim to pass the baking process together with the dough. Said water-based preparations have to endure the heat during the baking process without changing the shape or volume. The cacao pod husk derived pectin of the invention can be used as an agent for the improvement of baking stability of various water-based preparations, such as, but not limited to jams, jellies, jelly coatings, fillings, and the like.

Furthermore, the cacao pod husk pectin of the invention showed, in some examples, a pseudoelastic gelification when used in the concentration of 0.01-25.0 wt % in the finished product.

The cacao pod husk derived pectin of the invention can be used as a texturizer in a cream comprising encapsulated probiotics. Said pectin creates a texturizing, smoothening effect without harming the survival of the probiotic bacteria in the composition. Said pectin is used in the concentration range of 0.01-50.00 wt % in the finished product.

The cacao pod husk derived pectin of the invention can be used as an agent for improving the mouthfeel of instant mixes. The drinks are characterized by a fullness, nice mouthfeel and slightly higher thickness than a standard drink, when said pectin is used in the concentration range 0.01-25.0 wt % in the finished product.

In a final aspect, the cacao pod husk derived pectin of the invention is used in food products, preferably as an additive or substitute for plant-based meat or regular meat, more preferably in plant-based meat or in regular meat. In an embodiment, the cacao pod husk derived pectin of the invention is used in vegetarian burgers, preferably 1.0-30.0 wt % of the burger is cacao pod husk derived pectin, more preferably 2.0-10.0 wt %. In an embodiment, the cacao pod husk derived pectin of the invention is used in meat burgers, preferably 1.0-30.0 wt % of the burger is cacao pod husk derived pectin, more preferably 2.0-10.0 wt %. In an embodiment, pectin derived from dried cacao pod husk is used in vegetarian burgers, preferably 1.0-30.0 wt % of the burger is said pectin, more preferably 2.0-10.0 wt %. In an embodiment, pectin derived from dried cacao pod husk is used in meat burgers, preferably 1.0-30.0 wt % of the burger is said pectin, more preferably 2.0-10.0 wt %. In an embodiment, fresh husk derived pectin is used in plant-based meat or in regular meat, preferably 1.0-30.0 wt % of the meat is said pectin, more preferably 2.0-10.0 wt %. In an embodiment, dried husk derived pectin is used in plant-based meat or in regular meat, preferably 1.0-30.0 wt % of the meat is said pectin, more preferably 2.0-10.0 wt %. In an embodiment, 1-5 wt % or 2-10 wt % of dried husk derived pectin are added to vegan burger on top of the standard recipe to make vegan burgers. In an embodiment, 1-30 wt %, preferably 2-10 wt %, of fresh husk derived pectin are added to vegan burger on top of the standard recipe to make vegan burgers.

In food products, fresh husk derived pectin can be added as a stabilizer. Due to the high WHC, it is very suitable to be used in food products, it gives a nice color to the product, reduces baking losses and results in a firm product with a pleasant firmness. Pectins from fresh cacao husk in products can improve the mouthfeel, color, moisture content and texture. Drying of cacao husks improves the long-term storage stability however, the quality of the pectins is unexpectedly influenced. Pectins from dried cacao husks showed worse results for most parameters.

In an embodiment, baking yield was determined according to Twarogowska et al. (2022). The analyses are carried out in triplicate for each preparation. In an embodiment, the firmness of the baked burgers is determined using a texture analyzer. This parameter is expressed as the maximum force (N) to penetrate the sample by a means of a cylindrical probe (Ø=12 mm, speed=1 mm/s, depth of penetration=15 mm). Measurements were performed 4 times for each preparation.

The cacao pod husk derived pectin of the invention can be used as an elastic agent in food, cosmetics and pharmaceutical products. Said pectin, when used in concentration range 0.01-25.0 wt % in the finished product, shows an elastic effect which is not obtainable by using standard stabilizers. The cacao pod husk derived pectin is particularly suitable for cold drinks, flans, panna cotta, puddings and the like, as well as for non-food products such as gels and creams. The shear thinning behavior of the pectins results in the desired viscosity, feel and texture when mixed in these products.

The invention is further described by the following non-limiting examples which further illustrate the invention, and are not intended to, nor should they be interpreted to, limit the scope of protection.

### EXAMPLES

The present invention will now be further exemplified with reference to the following example(s). The present invention is in no way limited to the following examples, or preferred embodiments stated in the text. On the contrary, disclosed products, methods and applications according to the present invention may be realized in many different ways without departing from the scope of invention.

### Example 1

### Cacao Pod Husk Derived Pectin

The composition and characteristics of several cacao pod husk derived pectins was analyzed (Table 1). The degree of acetylation was for all samples between 20 and 60 wt % of dry extract. The degree of methylation was for all samples between 10 and 30 wt % of dry extract. The galacturonic acid content was at least 40 wt % of ash-free dry matter. The highest galacturonic acid content measured was 65 wt % of ash-free dry matter. The protein content, expressed in wt %, was higher for pectins derived from fresh husk (7-9 wt %) compared to pectins derived from dry husk (3-5 wt %). Both the water holding capacity and the molecular weight distribution were lower for the pectins derived from dry husk compared to fresh husk derived pectins.

TABLE 1
Characteristics of cacao pod husk derived pectins

| | Degree | Degree | | | |
|---|---|---|---|---|---|
| | of | of | Galact- | Water | Molecular |
| | acety- | methyla- | uronic | holding | weight >200 |
| | lation | tion | acid | capacity | kDa |
| Pectin | (%) | (%) | (wt %*) | (g/g) | (wt %) |
| Fresh cacao pod | 37 | 21 | 60 | 21 | 44 |
| husk derived | | | | | |
| pectin 1 | | | | | |
| Fresh cacao pod | 53 | 28 | 55 | 25 | 49 |
| husk derived | | | | | |
| pectin 2 | | | | | |
| Fresh cacao pod | 31 | 11 | 43 | 23 | 40 |
| husk derived | | | | | |
| pectin 3 | | | | | |
| Fresh cacao pod | 51 | 15 | 65 | 34 | 58 |
| husk derived | | | | | |
| pectin 4 | | | | | |
| Fresh cacao pod | 53 | 19 | 49 | 25 | 54 |
| husk derived | | | | | |
| pectin 5 | | | | | |
| Fresh cacao pod | 36 | 22 | 52 | 28 | 42 |
| husk derived | | | | | |
| pectin 6 | | | | | |
| Fresh cacao pod | 37 | 17 | 48 | 25.5 | 55 |
| husk derived | | | | | |
| pectin 7 | | | | | |
| Dry cacao pod | 34 | 22 | 54 | 7 | 10 |
| husk derived | | | | | |
| pectin 1 | | | | | |
| Dry cacao pod | 32 | 19 | 44 | 4 | 5 |
| husk derived | | | | | |
| pectin 2 | | | | | |
| Dry cacao pod | 35 | 20 | 32 | 5.2 | 42 (>100) |
| husk derived | | | | | |
| pectin 3 | | | | | |

*calculated on an ash-free dry matter basis

All further examples were conducted with Dry cacao pod husk derived pectin 3 and Fresh cacao pod husk derived pectin 7. Even a less purified (not washed) fresh husk pectin with only 48 wt % galacturonic acid calculated on an ash-free dry matter basis is performing superior vs benchmarks with a galacturonic acid content of 65 wt % of ash-free dry matter.

### Example 2

### Use the Fresh Cacao Pod Husk Derived Pectin as Stabilizer in Yogurt Preparation

Fresh cacao pod husk derived pectin was used to confirm protein dispersion stabilization at pH 5.0 in the acidic milk beverage (yoghurt). Commercially available apple pectin was used as a reference stabilizer in the reference sample. Dried cacao pod husk derived pectin was used as a second reference stabilizer in another reference sample.

After mixing 20 parts of a 1% stabilizing solution containing either cacao pod husk derived pectin or commercially available apple pectin, 10 parts of a 35% sugar solution and 20 parts of an 8% powdered skimmed milk solution, the mixture was cooled down and 50% citric acid solution was added in drops to adjust the pH value at 5.0. A homogenizer was used for homogenization of yoghurt samples at 150 kgf/cm2.

The yoghurt samples containing cacao pod husk derived pectin as the stabilizer were confirmed to exhibit protein dispersion stabilization at pH 5.0, which is above the isoelectric point of milk protein (pH 4.5). In addition, the viscosity of the yogurt was high and the beverage was characterized by a full body texture.

The yoghurt drink obtained using fresh or dry cacao pod husk derived pectin was stable, without visible aggregation or dividing of phases, without any kind of liquid collection on the top of the yoghurt surface. Yogurt is thick and spoonable rather than tender and pourable. However, the fresh cacao pod husk derived pectin yoghurt showed a lighter color, significant higher viscosity results and a higher texturizing performance compared to the dry cacao pod husk derived pectin yoghurt.

The yoghurt sample that contains commercially available pectin as the stabilizer exhibited unsatisfying stabilization of the reference dispersion. The viscosity was high and the texture was lumpy and gelatinous, differing substantially from the sample prepared using the cacao pod husk derived pectin of the invention.

### Example 3

### Viscosity of Pectin/Water Mix

To assess the thickening (pasting) and gel-forming properties of fresh cacao pod husk derived pectin an experiment was conducted and compared to a reference using dry cacao pod husk derived pectin. Different pectin/water mixtures (concentration 1%, 5%, 10%, 15%, 20%) were heating to 95° C. and subsequently cooled. The viscosity of said pectin/water mixtures was measured at a constant shear of 160 rpm.

The viscosity of fresh husk derived pectins was at least two times higher compared to dried husk at all concentrations and temperatures (Table 2). Pectin from fresh husk had similar thickening behavior to carrageenan at the same concentration of 1%. Calcium ions increased the viscosity of pectin extract by 35% for dried husk and 7% for fresh husk. At 1% addition fresh husk pectin performs similar to carrageenan. However, at the 5% addition level fresh husk pectin is 10 times higher at 20° C. (start), 7 times higher at 95° C. and 7 times higher at 20° C. (end of the measurement) than the dried husk pectin.

TABLE 2
Thickening behavior (cold and hot viscosity at constant shear)

| | Viscosity, cP | | |
|---|---|---|---|
| | Cold | Hot | Final |
| Pectin extract derived from dry husk 1% | 16 | 13 | 20 |
| Pectin extract derived from fresh husk 1% | 60 | 32 | 59 |
| Carageen 1% | 59 | 26 | 57 |
| Citrus pectin benchmark 1% | 6 | 4 | 8 |
| Apple pectin benchmark 1% | 14 | 7 | 20 |
| Pectin extract derived from dry husk 5% | 130 | 51 | 152 |
| Pectin extract derived from fresh husk 5% | 1402 | 378 | 969 |
| Citrus pectin benchmark 5% | 75 | 66 | 191 |
| Apple pectin benchmark 5% | 299 | 81 | 412 |

### Example 4

Pectins were mixed with distilled water using a magnetic stirrer to obtain a 5% mixture. Rheology measurements were performed using an Anton Paar Rheometer MCR 102. Parallel plates (PP50) were used at a gap of 0.5 mm. The viscosity was measured using rotational rheometry over the shear rate range 0.1-100/s at 20° C.

To compare the pectins extracted from cacao husk, two benchmark pectins were analyzed in parallel. The first benchmark pectin is a low methoxyl-weight pectin obtained from citrus. The degree of methylation is 7 wt %, the pH is 4.5 and the galacturonic acid content is at least 65 wt % of ash-free dry matter. The second benchmark pectin is a pectin obtained from apple. The degree of methylation is 34 wt %, the pH is 4 and the galacturonic acid content is at least 65 wt % of ash-free dry matter. Said benchmarks were also use in other examples.

The pectin extract derived from fresh husk dissolved in water showed the highest viscosity across all samples (Table 3). With increasing shear rate, the viscosity for all samples decreased. The absolute and relative difference between high and low shear rates, was significantly higher for the fresh husk derived pectin sample.

TABLE 3
Results from the viscosity measurements at 5% mixed with water

| | | | Pectin extract | Pectin extract |
|---|---|---|---|---|
| | Citrus pectin | Apple pectin | derived from | derived from |
| Shear | benchmark | benchmark | fresh husk | dry husk |
| rate, /s | Viscosity, cP | | | |
| 1 | 55 | 751 | 5355 | 231 |
| 1.58 | 53 | 746 | 5006 | 234 |
| 2.51 | 51 | 735 | 3837 | 217 |
| 3.98 | 50 | 725 | 3028 | 196 |
| 6.31 | 48 | 711 | 2470 | 181 |
| 10 | 47 | 694 | 2022 | 168 |
| 15.8 | 45 | 672 | 1629 | 157 |
| 25.1 | 44 | 644 | 1291 | 146 |
| 39.8 | 43 | 610 | 1008 | 135 |
| 63.1 | 42 | 570 | 779 | 124 |
| 100 | 41 | 522 | 599 | 112 |

### Example 5

### Fresh Cacao Pod Husk Derived Pectin as Lecithin Replacer in Chocolate

Test mixture was made as a mixture of 15 kg of cacao bean powder with 7.9 kg cocoa liquor, 25.3 kg cocoa butter, 50.3 kg white sugar, 25 kg whole milk powder, 17.5 kg low fat milk powder was mixed with 0.15 kg of the cacao pod husk derived pectin of the invention.

As the reference mixture, chocolate containing the same amount of ingredients, 15 kg of cacao bean powder with 7.9 kg cocoa liquor, 25.3 kg cocoa butter, 50.3 kg white sugar, 25 kg whole milk powder, 17.5 kg low fat milk powder 0.15 kg lecithin was produced. Both mixtures were kneaded and then worked in a 5-cylinder refiner.

The resulting masses were both subjected by conching and then treated like customary chocolate. Complete liquefication of the test chocolate mixture was performed by adding 8.9 kg cocoa butter and 0.4 kg cacao pod husk derived pectin.

Complete liquefication of the reference mixture was completed by adding 8.9 kg cocoa butter and 0.4 kg lecithin during conching. The resulting liquid chocolate mixtures (test and reference mixtures) were tempered and molded in the devices for normal chocolate.

The chocolate end product made with fresh cacao pod husk derived pectin was very pleasant in taste and quality, as evaluated by panelists (15). Its texture and taste were not significantly different from the reference mixture chocolate containing lecithin, as evaluated by panelists.

### Example 6

### Emulsion Formulation:

Oil-in-water emulsions were formulated. Each emulsion was formulated using the same method, whereby the aqueous phase was first prepared by dissolving the cacao-derived ingredient (15 g) in water (285 g), one day before emulsion formation. On the day of emulsion formation, all prepared aqueous solutions were re-agitated using a magnetic flea and stirrer plate to re-disperse any sedimented solids.

Oil-in-water emulsions were then prepared by homogenizing the oil phase (200 g rapeseed oil) with the aqueous phase using a high shear mixer (Silverson) at a defined time and energy input at ambient temperature. The processing parameters adopted consisted of using a Silverson shear speed of 10 230 rpm and a 9-minute mixing time, with the oil addition being carried out during the first 7-minutes. To prolong the shelf-life of the emulsion samples and to prevent microbial growth, 0.01% (0.05 g) of the antimicrobial agent sodium azide was added to each emulsion immediately after production.

### Example 7

### Emulsion Small Deformation Oscillatory Rheological Measurements

The viscoelastic properties of the five oil-in-water emulsions samples, prepared according to example 5, were measured by performing an amplitude strain sweep test. This test is conducted using an Anton Paar MCR 102 rheometer, fitted with a cone and plate geometry (diameter 50 mm and gap of 0.1 mm). The applied strain used was 0.01 to 100%, which was ramped from low to high, logarithmically increasing 5 points per decade (21 points in total). A constant frequency of 1 Hz and a measurement temperature of 20° C. were applied. The measurements were performed in duplicate on each emulsion sample after one day post-production.

The storage modulus is a measure of the stiffness of the material under test with a higher value being indicative of greater stiffness compared to a lower value. The elastic properties are a result of interactions between the different components in the emulsion sample and a greater number of interactions or stronger interactions lead to a higher storage modulus and hence a stiffer emulsion product.

The results (Table 4) show the surprisingly much higher storage modulus at 1% strain amplitude for fresh husk derived pectins compared to the other samples, including dry husk derived pectins. A storage modulus of 70-120 Pa measured for fresh husk derived pectins is very high.

Of the benchmarks, xanthan, carrageenan and the locust bean gum gave emulsions with large storage modulus indicating a significant amount of structure. This is to be expected as the primary function of these ingredients is viscosity modification. An increased viscosity provides a more viscous continuous phase and therefore a more stable emulsion by limiting the mobility of the oil particles. Of the Pectin benchmarks, the apple pectin formed a stable emulsion with small oil particles, however, the citrus pectin formed an unstable emulsion with larger particles. The soy lecithin benchmark showed almost no emulsion stiffness.

TABLE 4
Storage modulus measurements of the products in a 5 wt.
% 40:60 (o/w) emulsion after one day post-production

| Product | Storage modulus (Pa) |
|---|---|
| Apple pectin benchmark | 8.6 |
| Citrus pectin benchmark | 2 |
| Xanthan gum benchmark (9038) | 27 |
| Carrageenan benchmark (6530) | 48 |
| Locust bean gum benchmark | 27 |
| Soy lecithin benchmark | 0.02 |
| Pectin extract precipitated, from dry husk | 7.6 |
| Pectin extract precipitated, from fresh husk | 101 |

### Example 8

### Color of the Pectins

Pectin color plays an important role in the final appearance of the product to which it is added, and pectin extraction should not affect the color of the final product. Therefore, the pectin purification process is essential to remove any pigments that interfere with this quality parameter.

The luminosity parameter L indicates that dry husk derived pectin is darker than fresh husk derived pectin (Table 5). Values of a b and c were a somewhat higher for dried husk when compared to those of the fresh husk derived pectin. The delta-E value between fresh husk derived pectin and dry husk derived pectin, was 18.7. A Delta-E over 5 indicates a noticeable difference to the human eye. Therefore, color differences could be observed. Fresh husk pectin is significantly lighter compared to dried husk pectin, as caused by the difference in feedstock.

TABLE 5
Color scale measurements according to the CIELAB scale

| Parameter | Dried husk pectin | Fresh husk pectin |
|---|---|---|
| L | 31.3 | 48.1 |
| a | 14.2 | 6.8 |
| b | 28.5 | 25.5 |
| C | 31.9 | 26.4 |
| h | 63.5 | 75.0 |

### Example 9

### Water Holding Capacity

Results of the water holding capacity of several pectins is shown in Table 6. Pectin extracted form fresh husk showed a very high water holding capacity. This product is capable to hold approximately 25 times its weight in water. This is much more compared to pectin extracted from dry husk or other tested products.

TABLE 6
Water holding capacity of different products

| | Water holding capacity |
|---|---|
| Product | (g/g) |
| Citrus fiber | 7.55 |
| Pectin extract precipitated, from dry husk | 5.2 |
| Pectin extract precipitated, from fresh husk | 25.5 |

### Example 10

### Skin and Sun Care O/W Type Cream

The suncare cream was obtained by mixing oil and water phases. The oil phase contained (wt %): fullerene (C 60; manufactured by Aldrich) 0.002, diethyl sebacate 15.0, paramethoxy octyl 5.0, stearyl alcohol 3.0, stearic acid 3.0, glyceryl monostearate 3.0, Liquid paraffin 3.0. The aqueous phase was composed of fresh cacao pod husk derived pectin 3.0, triethanolamine 1.0, EDTA 2Na 0.05, titanium dioxide 5.0, glycerin 5.0, antioxidant q.s. preservative in water 25.0. Additionally, an appropriate amount of perfume was added. Oil phase was carefully dispersed into the water phase and emulsified using a homogenizer. The emulsion was cooled using heat exchange air to obtain an O/W type cream.

Obtained cream showed good stability and appearance. Moreover, it was characterized by a good functionality, enhancing the natural skin feel.

Compared to the obtained cream from dried cacao pod husk derived pectin, a similar emulsifying performance, but stronger emulsion stiffness was observed.

It is believed that the present invention is not limited to the embodiments described above and that some modifications or changes may be added to the examples described without revaluing the appended claims.

### Example 11

### Stability of Emulsions

The stability of the emulsions at ambient temperature was determined by visual observation and monitored for five days after their preparation, as described in the previous examples. A stable emulsion is obtained by sufficient surface active substances and/or by a viscous continuous phase. The Emulsification stability is calculated by dividing the height of emulsified layer by the height of total liquid layer.

Emulsions based on both pectin extract from dry husk and from fresh husk showed a very good stability (Table 7). Lower stability was observed for the emulsions based on citrus pectin, the carrageenan benchmark (6530) and the locust bean gum benchmark. The soy lecithin benchmark showed a reduced stability after 5 days.

TABLE 7
stability of emulsions based on several products after 1 and 5 days

| | Stability | Stability |
|---|---|---|
| | after | after |
| Product | 1 day (%) | 5 days (%) |
| Apple pectin benchmark | 100 | 100 |
| Citrus pectin benchmark | 80 | 69 |
| Xanthan gum benchmark (9038) | 100 | 100 |
| Carrageenan benchmark (6530) | 86 | 82 |
| Locust bean gum benchmark | 82 | 72 |
| Soy lecithin benchmark | 100 | 58 |
| Pectin extract precipitated, from dry husk | 100 | 100 |
| Pectin extract precipitated, from fresh husk | 100 | 100 |

### Example 12

### Emulsion Particle Size Distribution Measurements

The emulsion droplet size distributions for the emulsion samples, was measured one day after preparation, obtained as described in the previous examples. The droplet size distribution results are determined as a volume percentage in each of one hundred size ranges from <0.5 μm up to >822 μm. The lecithin gave the smallest particles of all the benchmarks which explains the creamy texture observed of the emulsion. Similar results were obtained for cacao husk derived pectins. Table 8 shows an overview of the most numerous particle diameter for each of the samples. For the emulsion based on the locust bean gum benchmark, the largest particles were found.

TABLE 8
particle diameter of droplets in emulsions
based on several products

| | Most numerous |
|---|---|
| | Particle diameter |
| Product | (μm) |
| Apple pectin benchmark | <0.5 |
| Citrus pectin benchmark | 24 |
| Xanthan gum benchmark (9038) | 9.5 |
| Carrageenan benchmark (6530) | 70 |
| Locust bean gum benchmark | 133 |
| Soy lecithin benchmark | <0.5 |
| Pectin extract precipitated, from dry husk | <0.5 |
| Pectin extract precipitated, from fresh husk | <0.5 |

### Example 13

### Vegan Burgers

The functionality of different fiber fractions of cacao husk in vegan burger analogs was researched. A total of 3 different fractions was evaluated (Pectin from fresh husk, Insoluble fiber from dried husk, pectin from dried husk) by adding 2% on top of the standard recipe to make vegan burgers.

### Result Color

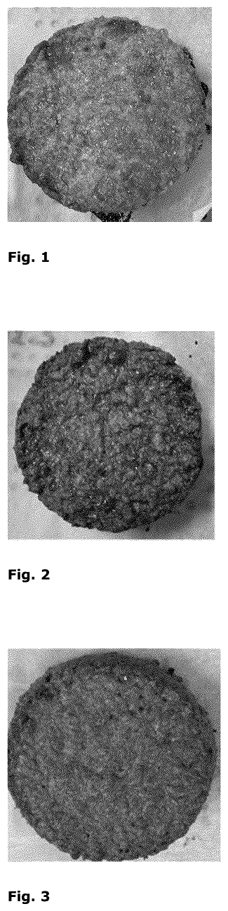

Addition of the different fiber fractions led to a more brownish color of the burgers compared to the standard vegan burger. The darker color was however not disturbing. On the contrary, it made the appearance of the burgers more similar to that of a meat burger. Especially the burger with the pectin fiber from fresh husk shows a high degree of resemblance with real meat. FIG. 1 shows an orange brown standard vegan burger. On the RGB scale the color corresponds with 163, 112, 45. FIG. 2 shows a burger with 2% insoluble fiber from dried husk added to the standard burger. This burger has a chocolate brown color and corresponds on the RGB scale with 145, 94, 68. FIG. 3 shows a burger with 2% pectin from fresh husk added to the standard burger, with a gingerbread brown color. On the RGB scale the color corresponds with 148, 92, 51.

### Results Baking Properties

Addition of the insoluble fiber from dried husk or pectin from dried husk lowered baking losses only to a small extent. On the other hand, addition of the pectin from fresh husk could lower the baking losses to a much higher extent. During the processing of this batch the latter already showed a higher viscosity compared to the other ones, which indicated a possible increase of the water binding of the finished burgers. Addition of the Insoluble fiber from dried husk had almost no impact on the firmness of the product. Addition of the pectin from dried husk and the pectin from fresh husk decreased firmness of the burgers. A soft and juicy product was obtained. The burgers were sufficiently firm not to fall apart into pieces when manipulating them. The burgers formed a coherent structure. Furthermore, after tasting them, the mouthfeel was described as similar to some meat products. Table 9 gives an overview of the baking losses. Pectins from fresh husk caused the burger to be more juicy and less dry with a pleasant firmness (Table 10).

TABLE 9
baking loss of burgers comprising different types of products

| Product | Baking loss (%) |
|---|---|
| Standard preparation | 33.3 |
| 2% addition of insoluble fiber from dried husk on top | |
| of standard preparation | 31.1 |
| 2% addition of pectin from fresh husk on top of | |
| standard preparation | 26.4 |
| 2% addition of pectin from dried husk on top of | |
| standard preparation | 31.7 |

TABLE 10
firmness of burgers comprising different types of products

| Product | Firmness (N) |
|---|---|
| Standard preparation | 24.6 |
| 2% addition of insoluble fiber from dried husk on top | 26.2 |
| of standard preparation | |
| 2% addition of pectin from fresh husk on top of | 3.5 |
| standard preparation | |
| 2% addition of pectin from dried husk on top of | 8.7 |
| standard preparation | |

### CONCLUSION

The pectin from fresh husk increases water binding and lowers firmness of the product. Furthermore, the pectin from dried husk also lowers the firmness while water binding is not affected. The overall coherence of the burgers is good. The burger with pectin from fresh husk showed the best color results, having a color close to meat burgers.

1. Fresh cacao pod husk derived pectin having a water holding capacity of at least 20.0 g water/g, wherein the fresh cacao pod husk has a moisture content above 50 wt %.

2. The fresh cacao pod husk derived pectin of claim 1 having a molecular weight >200 kDa for at least 40 wt % of the dry extract.

3. The fresh cacao pod husk derived pectin of claim 1 having-characterized in a storage modulus of 70 to 120 Pa measured in an emulsion consisting of 15 g pectin, 200 g oil, 285 g water at 20° C. and at a strain amplitude of 1%.

4. The fresh cacao pod husk derived pectin of claim 1 having a luminosity parameter L of at least 40.

5. The fresh cacao pod husk derived pectin of claim 1 having a viscosity of 4000 to 7000 cP at 20° C. in a 5% mixture with water at a shear rate of 1/s.

6. The fresh cacao pod husk derived pectin according to claim 1 and having two or more of the following characteristics:
(i) a molecular weight >200 kDa for at least 40 wt % of the dry extract,
(ii) a storage modulus of 70 to 120 Pa measured in an emulsion consisting of 15 g pectin, 200 g oil, 285 g water at 20° C. and at a strain amplitude of 1%,
(iii) a luminosity parameter L of at least 4,
(iv) a viscosity of 4000 to 7000 cP at 20° C. in a 5% mixture with water at a shear rate of 1/s.

7. The fresh cacao pod husk derived pectin according to claim 1 having:
a degree of acetylation of 30 to 50 wt % of dry extract;
a degree of methylation of 10 to 30 wt % of dry extract; and
a galacturonic acid content of at least 40 wt % of ash-free dry matter.

8. A method for the preparation of fresh cacao pod husk derived pectin according to claim 7, wherein the fresh cacao pod husk has a moisture content above 50 wt %: comprising a hot water extraction, wherein the pH upon completion of the extraction is between pH 3.0 and pH 5.0, and wherein the temperature for the hot water extraction is between 6° and 80° C.

9. Use of the fresh cacao pod husk derived pectin according to claim 7 as a dispersion stabilizer in a food product.

10. Use according to claim 9 of said fresh cacao pod husk derived pectin, wherein said food product is a protein food product, starch-containing food product or a confectionery product.

11. Use of the cacao pod husk derived pectin, according to claim 7, as an O/W or W/O emulsifier in food, cosmetic and pharmaceutical products.

12. Use according to claim 11 of said fresh cacao pod husk derived pectin, wherein said cacao pod husk derived pectin is used in a concentration 0.05 to 50.00 wt %.

13. Use according to claim 11 of said fresh cacao pod husk derived pectin, wherein said food product is a confectionery product, preferably a chocolate product.

14. Use of said fresh cacao pod husk derived pectin according to claim 7, as a coating agent, elastic agent or texturizer in food, cosmetic and pharmaceutical products or probiotics.

15. Use of said fresh cacao pod husk derived pectin according to claim 7, in plant-based meat or in regular meat.
